(19) United States
(12) Patent Application Publication
(10) Pub. No.: US 2010/0121217 A1
(43) Pub. Date: May 13, 2010

(54) DEVICE FOR MEASURING CORE TEMPERATURE

(75) Inventors: Alexander Padiy, Eindhoven (NL); Amy Cheung, Eindhoven (NL); Olaf Such, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/517,586

(22) PCT Filed: Nov. 26, 2007
(86) PCT No.: PCT/IB07/54789

§ 371 Date: Jan. 13, 2010

(30) Foreign Application Priority Data
Dec. 6, 2006 (EP) 06125479.3

(51) Int. Cl. A61B 5/01; G01K 3/00; G01N 25/18

(52) U.S. Cl. 600/549; 374/110; 374/44; 374/E03.001

(57) ABSTRACT

The invention provides a device and system for measuring core (11) body temperature, comprising two pairs of temperature sensors (8-1a, 8-1b, 8-2a, 8-2b), with a structure (2, 3, 4, 5, 6, 7) therebetween, and a heat flux modulator (9) for changing the heat flux through one pair (8-1a, 8-1b) more than the heat flux through the other pair (8-2a, 8-2b). By measuring the temperatures for the two pairs of temperature sensors, the core (11) body temperature may be derived. This device allows more design freedom, and it is easier to manufacture and gives a more accurate core temperature.

The present invention generally relates measuring the core temperature of an object, such as a human or animal body. In particular, the present invention relates to a device for measuring a core temperature of an object, comprising a structure having a first side to be positioned against the object, and a second side substantially opposite said first side, a first and a third temperature sensor, positioned at a mutual distance and each arranged for measuring a local temperature at the first side, a second and fourth temperature sensor, positioned at a mutual distance and each arranged for measuring a local temperature at the second side.

Document U.S. Pat. No. 5,816,706 discloses an apparatus for determining the internal temperature, for application to an object to be measured. The device comprises two structures with known ratio of their respective thermal conductivities. By measuring the temperatures of both sides of each of the two structures, a core temperature of the object may be determined by solving a system of two coupled equations.

A disadvantage of this system is that its performance strongly depends on the ratio of the thermal conductivities. This ratio should be large for a reasonable accuracy, which often also implies that at least one thermal conductivity is rather low, which in turn implies a long measurement time and additionally might significantly block (or at least disturb) the natural heat flow from the measured body to the ambient. Furthermore, the ratio should be accurately known and be stable during a long time, and the two structures may not show a different change in conductivity when the temperature changes, or in the course of their lifetime as this gives rise to inaccuracies. Furthermore, producing the device with the two structures with a known and well-controlled conductivity ratio poses great manufacturing difficulties, especially in mass-production.

An object of the invention is to provide a device for measuring a core temperature of an object, that is more easy to manufacture and/or that gives a more accurate measurement result and/or does not disturb the natural heat flow from the measured body (at least in the switched off state.

At least one of the above objects is achieved according to the invention with a device for measuring a core temperature of an object, comprising a structure having a first side to be positioned against the object, and a second side substantially opposite said first side, a first and a third temperature sensor, positioned at a mutual distance and each arranged for measuring a local temperature at the first side, a second and fourth temperature sensor, positioned at a mutual distance and each arranged for measuring a local temperature at the second side, wherein the device comprises a modulator means for changing a local heat flux between the first and second temperature sensor to a different extent than a local heat flux between the third and fourth temperature sensor.

“Changing a local heat flux to a different extent” means that the heat flux between the first and second temperature sensor is changed while substantially not affecting the heat flux between the third and fourth sensor, or at least that the heat flux between the third and fourth sensor is changed to a much less extent. This change may be positive or negative.

By providing a modulator means for changing the heat flux, instead of a different structure with a different thermal conductivity, it is now possible to use e.g. single structure with a homogeneous conductivity, which is much better controllable and easily manufacturable. Furthermore, since the heat flux may be greatly changed by providing appropriate modulator means, the dynamic range of the device may be improved.

It is noted that document U.S. Pat. No. 6,886,978 discloses a core temperature measuring device comprising a single pair of temperature sensors, between which is sandwiched a thermal insulator, and having variable temperature heater. However, this device can only measure core temperature using measurements of (inter alia) time-derivatives of a temperature, which is inherently less accurate. The present invention just uses temperatures that are directly measured by two pairs of temperature sensors to obtain the required data for a core temperature reading.

Other advantages and aspects of the invention are disclosed in the dependent claims.

In particular, the first and second temperature sensor are thermally coupled with a first thermal conduction constant, wherein the third and fourth temperature sensor are thermally coupled with a second thermal conduction constant, while any and all combinations of one of the first and second with one of the third and fourth temperature sensor are thermally coupled with a thermal conduction constant that is at least ten times smaller than the smallest of the first and second thermal conduction constant. This is a further elaboration of there being two structures that substantially independently measure temperatures. In each case, the first and second sensors are coupled, and the third and fourth sensors are coupled, while the mutual coupling between other combinations is at least ten times smaller, preferably at least 100 times smaller.

Advantageously, the first and second thermal conduction constants are substantially equal. This offers the opportunity to make a device that is very easy to manufacture. For in this case it is possible to use exactly the same material and construction for both subdevices, i.e. for both parts of the device, one part being substantially between the first and second sensor, and the second part being between the third and fourth sensor. This is obviously very easily producable. It furthermore has the advantage that it is very likely that any changes in thermal conductivity will be similar for both subdevcies. This limits the influence thereof on the accuracy of the measurements.

In an embodiment, the first and second temperature sensor are substantially opposite each other, with respect to the structure. In another embodiment, the third and fourth temperature sensor are substantially opposite each other, with respect to the structure. In each case, this will mostly ensure that the respective pairs of temperature sensors are relatively close to each other. This ensures, or at least allows, that the thermal coupling between the sensors of each pair is strong, while the influence of the surrounding parts and in particular cross-talk with the other (pair of) temperature sensors is minimized.

In a special embodiment, the first and/or third temperature sensor may be placed at a distance from the first side, being thermally coupled thereto, and the second and/or fourth temperature sensor may be placed at a distance from the second side, being thermally coupled thereto, in each case by means of a thermally conducting element having high thermal conductance. Also, the modulator means may be placed at a distance from the structure of the device, being thermally coupled to it by means of a thermally conducting element having high thermal conductance. Advantageously, the good thermal conductors comprise a metal such as aluminum or copper, or another material such as graphite. At least a part of the structure, that surround the said thermal conductors, could be, and preferably is, made of a thermally insulating material, such as foamed plastic, Kapton™ et cetera.

In a particular embodiment, the modulator means comprises at least one heater and/or cooler. This is a very practical means of modulating the local heat flux. For accurate computation of the core temperature, the difference between the heat flux between the first and the second sensor and the heat flux between the third and the fourth sensor should be significant meaning that the fluxes preferably differ by at least 10%. Such a difference in fluxes is easy to achieve by using an appropriate heat flux modulator element, as will be described in the following.

In each case, each of the at least one heater and/or cooler is placed in the heat flow path from the measured body to the ambient that runs either substantially through the first and the second temperature sensors or through the third and the fourth temperature sensors. Such a heater or cooler could e.g. be positioned on the first side, the second side, or inside the structure. There could be provided a heater and/or cooler with a variable power, or more than one heater and/or cooler, in each case to be able to enhance the device robustness with respect to ambient conditions and thus increase its accuracy.

In a special embodiment, the cooler comprises a Peltier element, a variable heat sink, a fan and/or or an evaporator, preferably with an evaporation fluid container. A Peltier-element is a compact and powerful, well controllable cooler instrument, that does not require any flow of a medium. A fan and/or an evaporator, and in particular a combination thereof, is a very simple cooling device, with a relatively large cooling power, especially if use is made of an evaporation fluid that easily evaporates and/or has a high latent heat, such as ethanol or the like. A variable heat sink may comprise a heat sink body with a high thermal emissivity and movable shielding means that may switch between a position in which the heat sink body is substantially shielded from ambient and a position in which the heat sink body is exposed to ambient. The shielding means preferably comprise a thermally insulating material.

In an embodiment, the heat sink comprises at least a first heat sink part that is movable with respect to at least one of the second heat sink part and the structure. Preferably, the first heat sink part is rotatable or translatable, or both, such that a varying area of the second part is shielded from ambient, or both. This is a simple embodiment of a variable heat sink, which makes a variable contact with ambient and can thus sink variable thermal fluxes to ambient. This in turn allows different temperatures to be reached at the corresponding temperature sensor.

In an embodiment, the heater comprises a Peltier element, or a resistive heater. These are very effective and often very simple, compact and well controllable heaters.

In particular, the device comprises a SpO2 and/or StO2 measuring device, the heater comprising a heat producing element of said SpO2 and/or StO2 measuring device, in particular at least one LED, thermistor and/or integrated circuit. This is an advantageous combination of a core temperature measuring device and a blood or tissue oxygenation measuring device. Herein, advantageously use is made of the circumstance that these oxygenation sensors may comprise a source of radiation, which, if thermally coupled to the temperature sensor(s) of the device, may serve as modulator means. In particular, the SpO2 and/or StO2 measuring device comprises at least one light source, preferably at least one LED, and/or at least one radiation measuring device and/or integrated circuit (e.g. for processing the corresponding measuring signals), a heat production of each of which could be used as a modulator means.

In an embodiment, the modulator means comprises a means for changing at least one of the first and second thermal conduction constant. Preferably, the means comprises an actuator for changing a distance between the first side and the second side, more preferably comprising a pointed pin connected to one of the first and second sides and pointing towards an opposite one of the first and second sides. In this embodiment, the thermal coupling itself, within the device, is locally adjustable, i.e. for only one pair of temperature sensors, or for both pairs in different ways. In particular, by providing an actuator for changing a distance between the first side and the second side, the thermal coupling amy be adjusted. For example, a part of the structure could be made in- and deflatable, or comprise a (piezo-)electrical, mechanical etc. actuator. It could also comprise a pin of a good thermal conductor such as copper or aluminum, positioned and dimensioned such that the pin is thermally connected to one of the parts of the structure, i.e. to one of the temperature sensors, while the tip of the pin is at a certain small (or zero) distance from and points towards an opposite part or sensor. Slightly changing the distance, especially from a zero distance to a non-zero distance, will dramatically change the thermal coupling constant.

In a special embodiment, the structure comprises a member with a shape that is outwardly curved, and preferably the structure comprises a member with a shape that is outwardly curved, preferably such that the part where the first temperature sensor is present projects from the first side. In this way, the first temperature sensor will contact the object to be measured in a suitable way, and a reliable contact can be provided. In particular, a member may be provided for that function, unto which the first temperature sensor is attached or attachable. Preferably, a member is arranged to be able to exert a spring force or resilience that is able to press the first side, and thus the first temperature sensor, onto the object.

Thereto, advantageously, the member comprises a flexible material, preferably a spring, in particular a leaf-spring. Herein, “flexible” means that the shape is visibly alterable when exerting a normal force with a human finger. An advantage of the member being flexible is that for example changes such as movements in the object to be measured, in particular a human body (the skin), can be accommodated more easily.

Preferably, the member is of a substantially uniform thickness. In such a case, the heat flow will be more even in the structure, in particular in the member. This greatly simplifies the calculations, and allows relatively simple approximations to hold validly.

In an embodiment, the member is layered. Preferably the member comprises a layer of Kapton™ or neoprene, and/or comprises a layer of a good thermal conductor on at least one surface of the member. Such a layered structure allows an even temperature distribution at the first side and the second side of the member. Again, this simplifies heat flow and calculations and increases the accuracy of the temperature measurement. Herein, a thermal conductor is good if it has a thermal conductivity of at least 1 W/mK, and preferably comprises a metal layer. Furthermore, another layer, preferably a central layer, comprises a good thermal insulator, such as Kapton™ or neoprene, which combine a low thermal conductivity with desirable resilient properties. Other materials are not excluded.

In an advantageous embodiment, the device comprises a holding construction for holding the device in a stabile position onto the object. Although the device could also be useful without such a holder construction, for example by holding it manually in a desired position, its usefulness could be increased by providing such a holding construction. In that case the device could be left unattended and still perform its function reliably. In particular, the holding construction comprises side walls around the member and/or fixation means for fixating the device onto the object, more preferably comprising an adhesive layer and/or a strap. Such side walls may be advantageous to provide a pretension to the member, which is useful for establishing a reliable contact with the object. Furthermore fixation means preferably comprise an adhesive layer and/or a strap in order to fixate the device to the object. Of course, depending on the object, other fixation means may be contemplated.

In an embodiment, the device according to the invention further comprises a calculation unit, arranged to calculate the body core temperature from respective temperatures measured by the first through fourth temperature sensor. In suitable cases, the modulator means are active or activated, in order to have a difference in heat flux between the respective sets of temperature sensors. Although it could be sufficient for the device to be able to provide respective temperature readings, from which a skilled person or some external device could calculate the actual core temperature, providing a calculation unit in the device for performing this task is advantageous since it avoids such human or external calculation.

Although the skilled person could easily derive a formula for the core temperature, a simple example will be given in the description of the drawings, for background information.

On the basis of these or similar calculations, the device may also be arranged to calculate the body core thermal resistivity from respective temperatures measured by the first through fourth temperature sensor, or to relate the thermal resistivity of the measured body to physiological parameters, e.g. blood perfusion of the measured subject skin.

For each of the devices according to the invention, it is possible to provide an alarming device, that gives off an alarm signal if a temperature becomes too high or too low. This could relate to a measured body part temperature, or a temperature as caused by the modulator means, such as in the case of an overheated heater. The alarm signal may be visible, auditive, or a radio signal or the like to a more remote observer.

It is also an option to provide the device with communication means, such as an Internet connection or a radiotransmitter, to provide signals or a read out to a remote location.

The invention also relates to a temperature measuring system, comprising a plurality of devices according to the invention, preferably provided in a matrix structure.

The matrix structure, or grid, may be embodied as a casing or other member. Such a temperature measuring system could be advantageous in that at least one device will be positioned in a favourable spot on the body part for measuring core body temperature. In practice, a proper measurement location if the distance from the device to the core is minimized. Such a proper location could be found manually, but the system of the invention will automatically provide a plurality of systems in different locations, such that at least one device will be positioned close to the core. This device will give a relatively more accurate and quicker result.

Having explained the summary of the invention, some preferred but only illustrative and non-limiting embodiments will be shown in the drawings, in which:

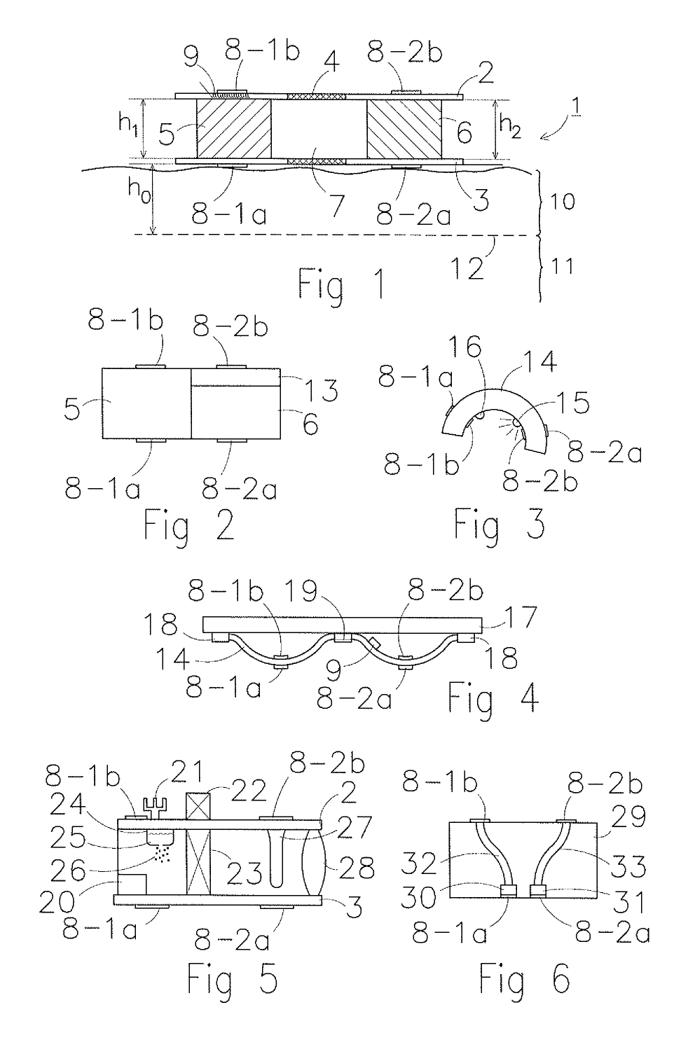

FIG. 1 very diagrammatically shows an embodiment of the device 1 of the present invention, in a side elevational cross-sectional view;

FIG. 2 diagrammatically shows a slightly different embodiment;

FIG. 3 diagrammatically shows another embodiment of the device, that could be used as an ear plug;

FIG. 4 diagrammatically shows another embodiment of the device according to the invention, in a side elevational cross-sectional view;

FIG. 5 diagrammatically shows a device according to the invention, with a large number of different modulator means; and

FIG. 6 diagrammatically shows another embodiment of the device according to the invention, in a cross-sectional view.

FIG. 1 very diagrammatically shows an embodiment of the device 1 of the present invention, in a side elevational cross-sectional view.

Herein, 2 and 3 denote a first and second structure part, respectively, with thermally insulating portions 4.

A first and second thermal insulators are denoted 5 and 6, respectively, with an insulating portion 7.

Also shown are first through fourth temperature sensors, 8-1a, 8-1b, 8-2a and 8-2b, respectively. A heater is denoted 9, while the device 1 is positioned on a body part with a skin 10 and a core 11, with a virtual interface 12 therebetween.

The four temperature sensors, which will collectively be denoted by 8 in the following, could be any suitable sensor, such as a thermocouple or the like. Two sensors are positioned on a skin side of the device 1, i.e. on or in structure part 3, and two sensors are positioned opposite, i.e. on or in structure part 2. Note that these structure parts 2 and 3 are optional, in case the sensors are placed directly onto or in thermal insulators 5 and 6. The structure parts 2 and 3 could also be made of a good thermal conductor, such as a metal, to ensure a homogeneous temperature at the respective sensor sides. In that case, thermally insulating portions 4 would be required, to prevent cross-talk between the sensors. The portions 4 could be made of e.g. foam, rubber, or other insulators.

Thermal insulators 5 and 6 could similarly be any thermal insulator such as foam, or various other plastics, or the like. They could be separated by another insulator, such as air, to save material and cost. Alternatively, and preferably, both insulators are one and the same body, with no portion 7 being present in between. The thermal resistivities of the insulators 5 and 6 should be stable and known, while the insulating portion 7 should have a high thermal resistivity that may be unknown and/or varying as long as it is much larger than the thermal resistivities of the insulators 5 and 6.

The heater 9 is positioned near sensor 8-1b, but could also be positioned near sensor 8-1a, or even in between those sensors. Note that the indication first through fourth is simply derivable from the position of the heater or cooler or other modulator means. In this case, the heater 9 is a simple resistive coil.

Although the principles of measuring core temperature by measuring various temperatures, and solving thermodynamic equations, some background will be given below.

The two thermal insulators 5 and 6 have respective thermal conductivities K₁ and K₂, and respective thicknesses h₁ and h₂. As noted above, these could be substantially equal. The skin is deemed a portion between the surface and a virtual interface, below which the temperature is deemed equal to the core temperature, with a thickness h₀ and a thermal conductivity K₀. Furthermore, the four sensors 8 measure respective temperatures T₁ₐ, T_{1b}, T₂ₐ and T_{2b}.

In the steady state, the heat fluxes in the left part of the device 1, i.e. from the top at sensor 8-1b to the bottom at sensor 8-1a is the same as theat from the bottom at 8-1a through the skin, and similarly for the right part. Herein, it will be assumed that T_{core} to be determined, as well as K₀ are the same below the whole of the device 1. After some simple mathematics, it follows that

$${T}_{core} = \frac{ξ ( {T}_{1 a} - {T}_{1 b} ) {T}_{1 b} - ( {T}_{2 a} - {T}_{2 b} ) {T}_{1 a}}{ξ ( {T}_{1 a} - {T}_{1 b} ) - ( {T}_{2 a} - {T}_{2 b} )}$$

$$where$$

$$ξ = \frac{{K}_{1}}{{K}_{2}} \cdot \frac{{h}_{2}}{{h}_{1}}$$

Preferably, the heater 9 should be operated such that the heat flux difference between left and right part is significant, such that also the difference in temperature differences left and right are significant, especially in the case where ξ=1.

FIG. 2 diagrammatically shows a slightly different embodiment.

Herein, as in all of the drawings, similar parts are denoted by the same reference numerals, and will generally not be described further. In the presently shown embodiment, part 13 denotes some additional device, such as a CPU, for example a calculation unit for calculating the core temperature. Such an additional device could also have a known or controllable power that could be used for heating, even though it is not a separate dedicated heater.

FIG. 3 diagrammatically shows another embodiment of the device, that could be used as an earplug. This device comprises a SpO2 and/or StO2 sensor.

Herein, 14 denotes a single continuous thermal insulator, while 15 denotes a light source such as a LED or LED combination, and 16 is an optical sensor.

The thermal insulator 14 could be a flexible member, dimensioned and shaped to be fitted into an ear, and able to press temperature sensors 8-1a and 8-2a into contact with an inner part of the ear. The insulator 14 could be made of e.g. Kapton™, neoprene or the like.

The light source 15 could be a light source suitable for measuring blood or tissue oxygenation such as a LED or LED combination that is able to emit e.g. red light, of two different wavelengths, or a sufficiently broad range of wavelengths. The sensor 16 is an optical sensor able to provide a signal corresponding to an intensity of reflected light. The sensor should be able to measure intensity at least the above mentioned wavelength(s), but is not particularly limited otherwise.

This embodiment is advantageous in that it not only measures the clinically important blood and/or tissue oxygenation, but also provides a core temperature with a higher accuracy than known ear-insertable temperature sensing devices that simply measure a surface temperature of an inner ear wall or a tympanic temperature.

FIG. 4 diagrammatically shows another embodiment of the device according to the invention, in a side elevational cross-sectional view.

Herein, 17 denotes a holding structure, 18 denotes a fixating structure, while 19 denotes a central fixator.

The insulator 14 is an insulating member that has an outwardly bulged shape, in order to provide good thermal contact between the sensors and the object to be measured. The insulator 14 could be resilient, such as certain rubbers, to improve contact even when the subject moves or changes shape otherwise.

The insulator is fixed onto holding structure 17 by means of fixating structure 18, that could e.g. simply be a clamp, adhesive et cetera. Central fixator 19 guides the insulator 14 therethrough. Alternatively, there could be provided two separate insulators 14, each fixed in the holding structure 17, or there could be provided an insulator 14 with a single outward bulge, the sensors 8-1a and 8-2a both being provided on the outside of the same bulge. The heater/cooler 9 could also be positioned as embedded in the holding structure 17. Heat flux from the body to the ambient will be also modulated in this case.

FIG. 5 diagrammatically shows a device according to the invention, with a large number of different modulator means. Each of these means may be provided separately or in any combination.

In this case, there is air between structure parts 2 and 3, which is however not necessary.

There is shown a Peltier element 20, a heat sink 21, a first fan 22, a second fan 23, a fluid container 24 with evaporation fluid 25, a cloud 26 of evaporated fluid, a thermally conducting pin 27 and an actuatable spacer 28.

The Peltier element is a compact and efficient cooler means or heater means. Note that cooler means could be advantageous in that skin is easily able to withstand temperatures that are e.g. up to 30° C. lower than core body temperature, at least during a short time and at a small area, while temperatures above 45° C. are experienced as painful, which would be less than 10° C. above core body temperature in most cases. Hence, a cooler means provides a larger dynamic range, and more noise-free and more accurate measurements.

The heat sink 21 is preferably a variable heat sink with some shielding means (not shown) to be able to provide two situations: a first in which the heat sink is passive in the sense that it cannot sink heat because it is shielded, and a second in which the shielding is removed and it can sink heat.

Alternatively, if the first fan 22 is provided, the heat sinking capacity of heat sink 21 can be changed by turning the first fan 22 on or off. The fan 22 could also provide a cooling power by itself.

The same holds for the second fan 23, that could provide a cooling air current through the device. In addition or alternatively, if a fluid container with evaporation fluid, such as water or alcohol or the like, is provided, evaporation of the fluid may sink heat through the latent heat needed for that evaporation. The second fan 23 can support this evaporation by blowing away the cloud 26 of evaporated fluid, such that evaporation is accelerated.

The pin 27 provides a thermal conductivity between sensors 8-2a and 8-2b that differs from that between 8-1a and 8-1b, and which can be varied by operating actuatable spacer 28. What is relevant is that the thermal conductivity of the material of pin, preferably a metal such as copper or silver, is higher than that of its surroundings, in this case air. The lines of thermal conduction then concentrate near the tip of the pin 27, and a small movement can change the effective thermal coupling significantly. Such a small movement may be brought about with the help of actuatable spacer 28, such as an inflatable device or a piezo-electrical device. Note that in this case one could call sensors 8-2a and 8-2b the first and second temperature sensor.

In an alternative embodiment, the evaporator 23, fluid container 24 and second fan 23 is positioned on the outside of the structure. This is often a much more practical solution, as in the embodiment shown in FIG. 5 one could have difficulties with getting rid of vapor out of the structure, and thus the evaporator could have problems with evaporation.

Peltier element 20 can be put on the outside as well as an alternative embodiment.

FIG. 6 diagrammatically shows another embodiment of the device according to the invention, in a cross-sectional view.

An insulating body is denoted by 29, while also shown are separate insulators 30 and 31 and thermal conductors 32 and 33.

Here, the sensors 8-1b and 8-2b are positioned relatively further apart than sensors 8-1a and 8-2a. This may for example be advantageous for adapting the device shape to the requirements of the application and creating the difference in heat fluxes from the different sensors to the ambient.

1. A device for measuring a core (11) temperature of an object, comprising
a structure (2, 3, 4, 5, 6, 7; 14; 29, 30, 31, 32, 33) having a first side to be positioned against the object, and a second side substantially opposite said first side;
a first and a third temperature sensor (8-1a, 8-2a), positioned at a mutual distance and each arranged for measuring a local temperature at the first side; and
a second and fourth temperature sensor (8-1b, 8-2b) positioned at a mutual distance and each arranged for measuring a local temperature at the second side;
wherein the device comprises a modulator means (9; 13, 15, 16; 20, 21, 22, 23, 24, 27, 28) for changing a local heat flux between the first (8-1a) and second (8-1b) temperature sensor to a different extent than a local heat flux between the third (8-2a) and fourth (8-2b) temperature sensor.

2. The device according to claim 1, wherein the first and second temperature sensor (8-1a, 8-1b) are thermally coupled with a first thermal conduction constant, wherein the third and fourth temperature sensor (8-2a, 8-2b) are thermally coupled with a second thermal conduction constant, while any and all combinations of one of the first and second with one of the third and fourth temperature sensor are thermally coupled with a thermal conduction constant that is at least ten times smaller than the smallest of the first and second thermal conduction constant.

3. The device according to claim 2, wherein the first and second thermal conduction constants are substantially equal.

4. The device according to claim 1, wherein the first and second temperature sensor (8-1a, 8-1b) are substantially opposite each other, with respect to the structure (2, 3, 4, 5, 6, 7; 14; 29, 30, 31, 32, 33).

5. The device according to claim 1, wherein the third and fourth temperature sensor (8-2a, 8-2b) are substantially opposite each other, with respect to the structure (2, 3, 4, 5, 6, 7; 14; 29, 30, 31, 32, 33).

6. The device according to claim 1, wherein the modulator means (9; 13, 15, 16; 20, 21, 22, 23, 24, 27, 28) is placed substantially on top of a stack that comprises the first and the second temperature sensors (8-1a, 8-1b) and the structure (2, 3, 4, 5, 6, 7; 14; 29, 30, 31, 32, 33) in between.

7. The device according to claim 1, wherein the modulator means (9; 13, 15, 16; 20, 21, 22, 23, 24, 27, 28) comprises at least one heater (9; 13, 15, 16; 20) and/or cooler (20; 21, 22, 23, 24, 27, 28).

8. The device according to claim 7, wherein the cooler comprises a Peltier element (20), a heat sink (21), a fan (22, 23) and/or or an evaporator, preferably with an evaporation fluid container (24).

9. The device according to claim 7, wherein the heater comprises a Peltier element (20), or a resistive heater (9).

10. The device according to claim 7, wherein the device comprises a SpO2 and/or StO2 measuring device (15, 16), the heater comprising a heat producing element of said SpO2 and/or StO2 measuring device, in particular at least one LED (15), thermistor (16) and/or integrated circuit.

11. The device according to claim 1, wherein the modulator means comprises a means for changing at least one of the first and second thermal conduction constant, preferably an actuator (28) for changing a distance between the first side and the second side, more preferably the device comprising a pointed pin (27) connected to one of the first and second sides and pointing towards an opposite one of the first and second sides.

12. The device according to claim 1, wherein the structure (2, 3, 4, 5, 6, 7; 14; 29, 30, 31, 32, 33) comprises a member (14) with a shape that is outwardly curved.

13. The device according to claim 12, wherein the member (14) comprises a flexible material, preferably a spring, in particular a leaf-spring.

14. The device according to claim 12, wherein the member (14) is of substantially uniform thickness.

15. The device according to claim 14, wherein the member (14) is layered, and preferably comprises a layer of Kapton™ or neoprene, and/or comprises a layer of a good thermal conductor on at least one surface of the member.

16. The device according to claim 12, comprising a holding construction (17) for holding the device in a stable position onto the object, preferably comprising side walls (18) around the member (14) and/or fixation means (18, 19) for fixating the device onto the object, more preferably comprising an adhesive layer and/or a strap.

17. The device according to claim 1, further comprising a calculation unit (13), arranged to calculate the body core (11) temperature from respective temperatures measured by the first through fourth temperature sensor (8).

18. The device according to claim 1, further comprising a calculation unit (13), arranged to calculate the body core (11) thermal resistivity from respective temperatures measured by the first through fourth temperature sensor (8).

19. The device according to claim 1, further comprising a calculation unit (13) that relates the thermal resistivity of the measured body to physiological parameters, in particular blood perfusion of skin (10) of the measure body part.

20. (canceled)

21. A plurality of devices according to claim 1, wherein the devices are provided in a matrix structure in a temperature measuring system.
